(19) United States
(12) Patent Application Publication
(10) Pub. No.: US 2012/0156689 A1
(43) Pub. Date: Jun. 21, 2012

(54) METHOD AND APPARATUS FOR DETERMINING AN ANALYTE PARAMETER

(75) Inventors: Keith Robert Willison, London (GB); David R. Klug, London (GB); Oscar Ces, London (GB); Edward Burgin, Nottingham (GB); Ali Salehi-Reyhani, Gwent (GB); Joseph John Kaplinsky, London (GB)

(73) Assignee: IMPERIAL INNOVATIONS LIMITED, South Kensington (GB)

(21) Appl. No.: 13/384,009

(22) PCT Filed: Jul. 14, 2010
(86) PCT No.: PCT/GB2010/001343

§ 371 Date: Mar. 7, 2012

(30) Foreign Application Priority Data
Jul. 14, 2009 (GB) 0912231.8

(51) Int. Cl. G01N 33/53; B01L 3/00; C12M 1/34; G01N 21/64

(52) U.S. Cl. 435/7.2; 436/501; 435/7.1; 422/502; 422/69; 435/287.2

(57) ABSTRACT

A method of determining an analyte parameter is described for an analyte in a detection region having at least one analyte binding zone. The method includes detecting presence of an analyte element bound at the binding zone, incrementing or decrementing a count and preventing recount of the element to obtain absolute quantification.

The invention relates to a method and apparatus for determining an analyte parameter.

Various analysis systems exist for detecting single molecules on microarrays. These approaches use low probe density arrays and single molecule spectroscopy. For example in patent publication WO02/074988 a small percentage of analyte such as DNA is captured and single molecules are visualised using optical techniques.

Known systems, however, suffer from various problems including an inability to distinguish between a specifically bound analyte and a randomly diffusing analyte, limitation to high copy number proteins and high protein concentration samples and inability to absolutely quantify the captured analyte.

The invention is set out in the claims. By detecting the presence of an analyte element bound at a binding zone and preventing the recount of said element, an absolute number of analyte elements can be obtained. Furthermore, where the analyte is processed in a confined region such as a microfluidics region, the majority of the analyte can be bound and detected in the mass assay regime.

Embodiments of the invention will now be described, by way of example, with reference to the drawings of which:

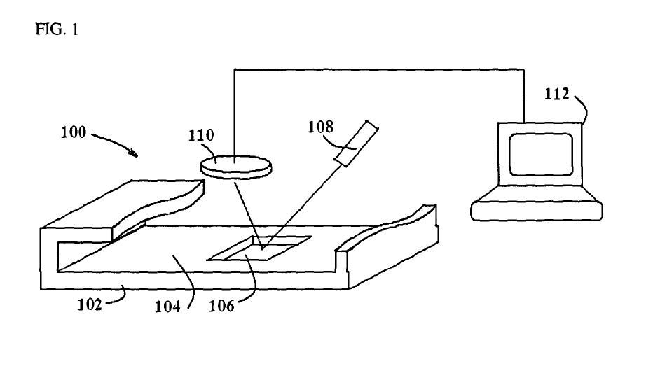

FIG. 1 is a schematic diagram showing an apparatus for determining an analyte parameter according to the present invention;

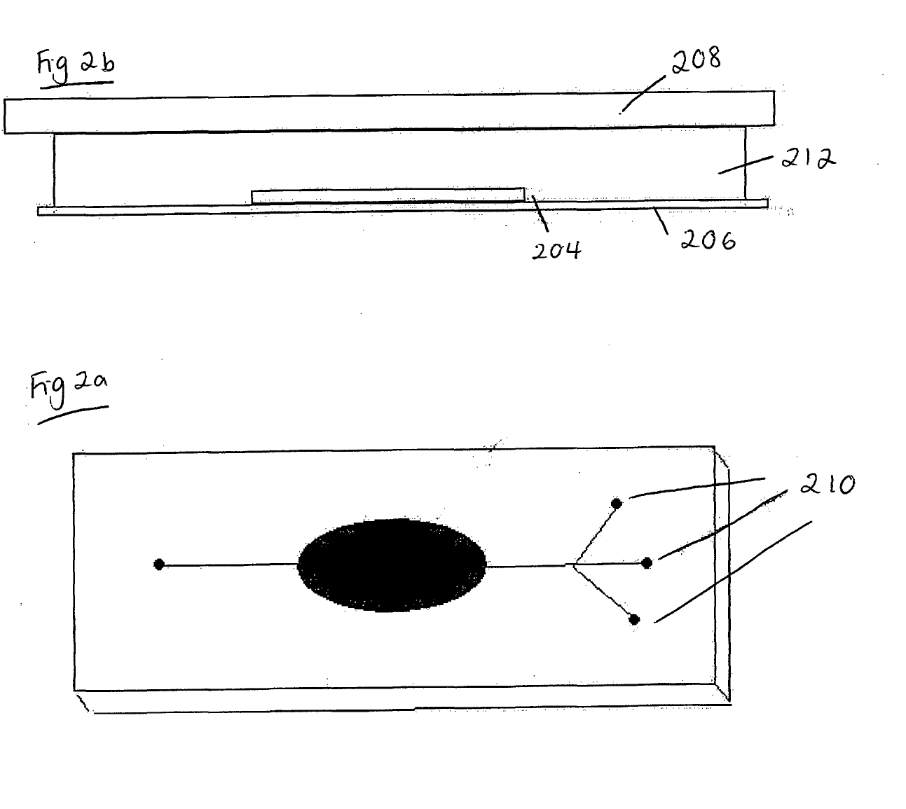

FIGS. 2a and 2b are detailed diagrams of an embodiment of the invention;

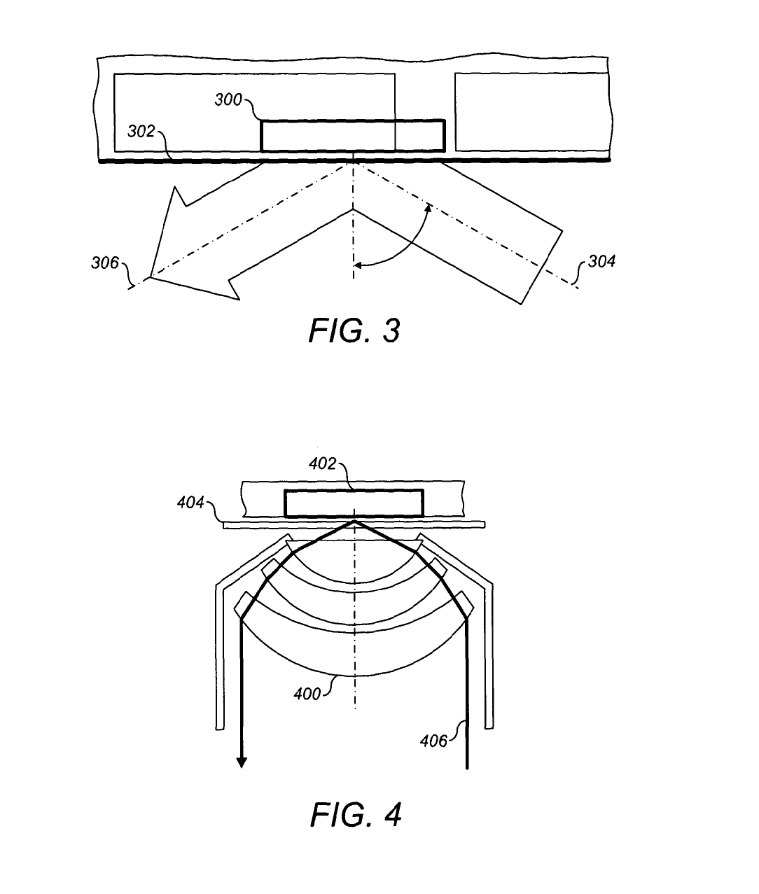

FIG. 3 shows incident and reflected light on a sample;

FIG. 4 shows incident and reflected light using an objective;

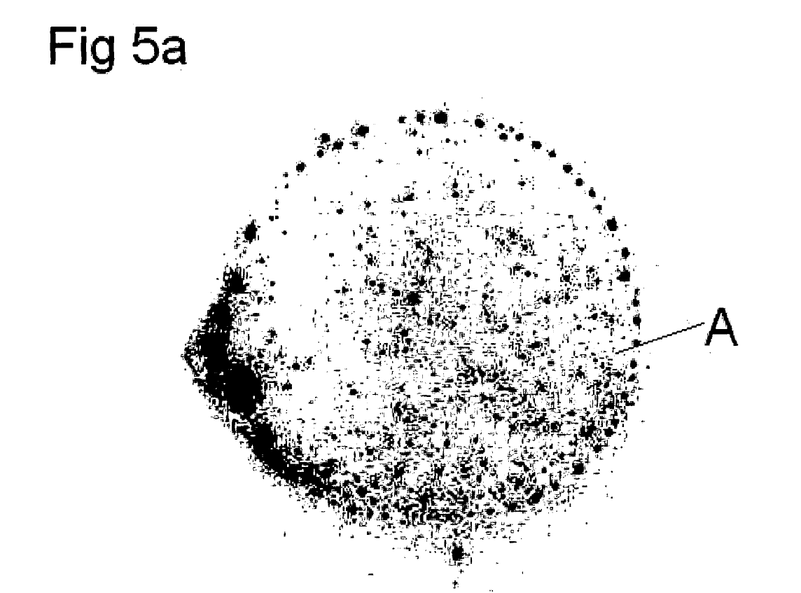

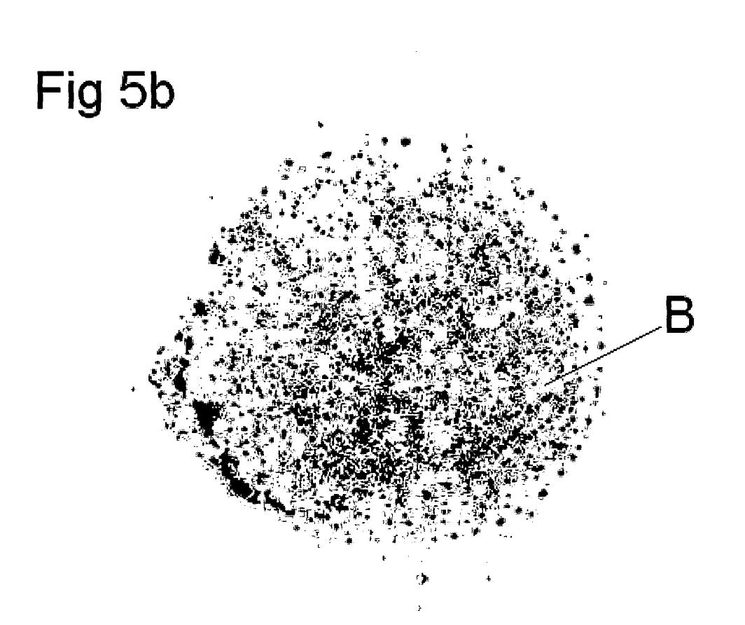

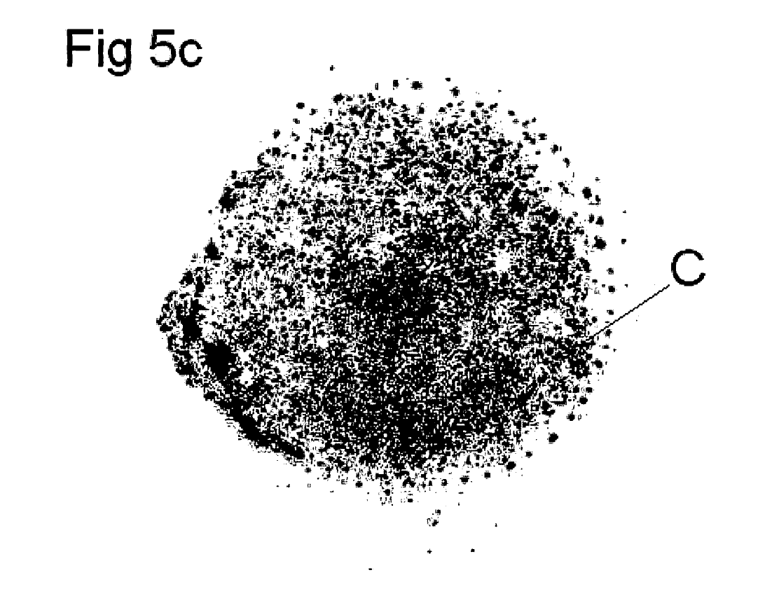

FIGS. 5a to 5c show sequential image frames;

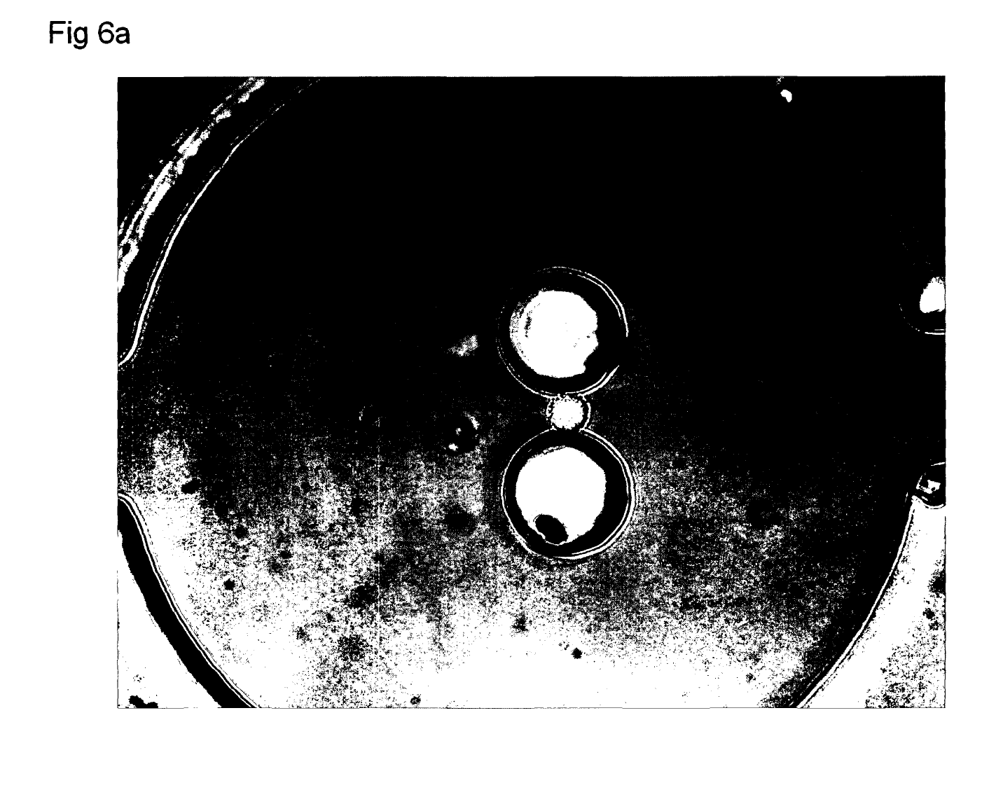

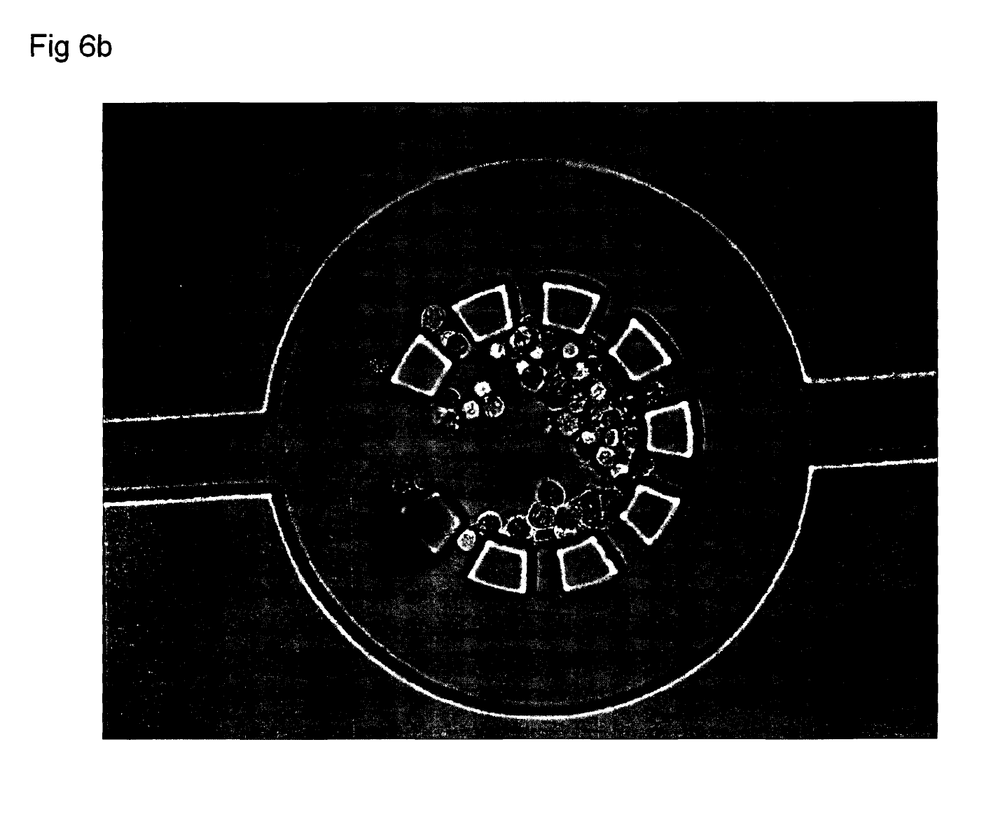

FIG. 6a shows a single cell trapped in a geometry which permits the stable trapping of a single cell;

FIG. 6b shows multiple cells trapped in a geometry which permits the stable trapping of many cells;

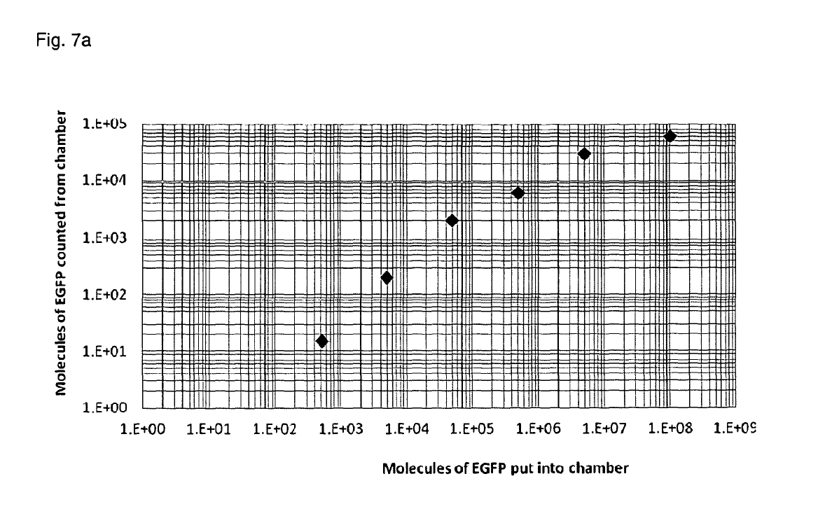

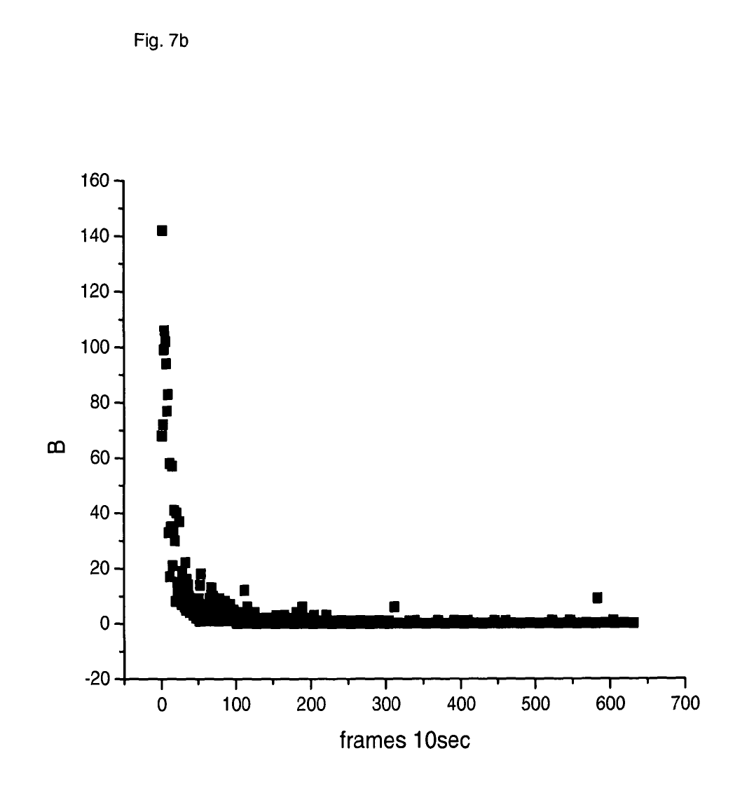

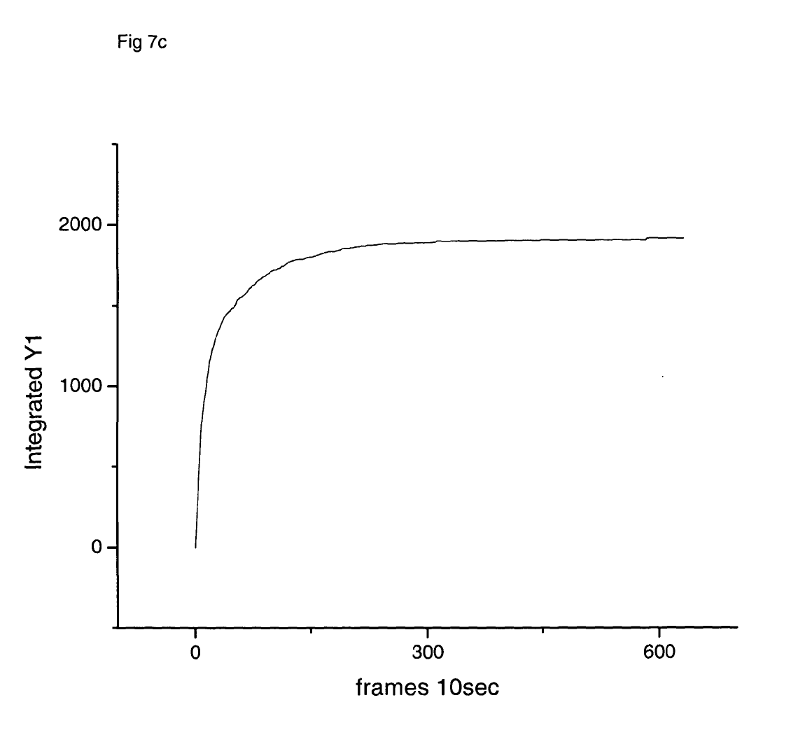

FIG. 7a is a trace showing the number of molecules counted as a function of time, in an experiment using single molecule bleaching;

FIG. 7b shows the number of molecules decremented in each frame as compared with the previous frame (Y axis) over a succession of frames ten seconds apart, for the experiment of FIG. 7a; and

FIG. 7c shows the running total of the number of molecules counted for the experiment of FIGS. 7a and 7b over a succession of frames.

In overview, an analysis apparatus such as a biosensor is provided allowing single molecule counting at high sensitivity and providing absolute quantification. Referring to FIG. 1, an analysis apparatus shown generally at 100 comprises a detection region for example in a microfluidic device 102 having an analyte receiving chamber for example a static hybridisation chamber 104. One or more binding zones, for example affinity patches 106, are provided comprising for example antibodies, DNA probes or lectins. The presence of an analyte element such as a single molecule bound to the affinity patch is detected by a detector for example including an excitation element such as a laser 108 inducing fluorescence and a detection element such as a camera 110. A count is implemented for each detected molecule at a processor 112. After each count a recount of the molecule is prevented. For example this can be achieved by irreversibly bleaching the molecule when it is counted, or by time multiplexing the detected image at the camera 110 and subtracting from the sequences of images molecules already detected. Multiple different biological molecules can be quantified for example using affinity patches with different binding characteristics. The device may be movable in the horizontal plane using an X or XY movable motion stage (not shown) allowing spatial scanning.

It is thus possible to determine the amount of analyte in a fixed and static system, without having all the analyte bound at equilibrium. With knowledge of the number of antibodies for example in each affinity patch and the static volume of the chamber it is in, the rate at which the analyte binds to the sensing surface and the amount at or near equilibrium provides information on the amount of sample in the static chamber. Alternatively, by counting each molecule as it binds to the surface and irreversibly bleaching this molecule (loss of fluorescence), so that it will not show again over time, each molecule will have been bound to the affinity patch, counted and irreversibly bleached; thus yielding the absolute number of analyte molecules in the sample.

The confined and static hybridisation techniques can be applied to fluorescently labelled analytes or can rely on any inherent detection property of the analyte as appropriate. The detection technique may rely for example on evanescent low incident angle light causing the fixed and immobilised substances to emit in the hybridisation chamber—a non-bound analyte will not affect the count as it will have a low dwell time in the vicinity of the binding zone. Because the approach can be adopted in the mass transfer regime using a small, for example microfluidic, volume, the majority of the analyte can be bound and detection can be performed over a period of time enhancing the absolute quantification techniques.

Turning to the nature of the apparatus in more detail, a plan view is shown in FIG. 2A and a sectional cut through side view is shown in FIG. 2B. The arrangement includes a chamber 204 which is a static hybridisation chamber in the embodiments shown but alternatively could be a dynamic flow chamber as required. The chamber can be, for example, approximately 6 mm squared in diameter and 100 micrometres high. It can be fed by sample inlets 210. The chamber can be closed by a slide 208 for example of 1 mm thickness, a 100 micrometre thick cover glass 206 binding the other surface and spaced by, for example, 4 mm thick soft polymer pdms 212. Through the application of microfluidics, it is possible to confine the analyte in static hybridisation chamber 204. The chamber can have one or more sensing surfaces 206, comprised of either glass or quartz coverslips with affinity agents such as antibodies immobilised in specific predefined locations. Single cells may be selectively or passively trapped (either optically or hydrodynamically in a microfluidic or flow device) to a defined coordinate or region. This coordinate or region may be co-localised with an antibody patch or other affinity patch. The cell (or cells) may then be lysed using laser induced microcavitation for example with 532/1064 mm ns-pulsed radiation commonly referred to as laser lysis or other lysis techniques such as optical or chemical techniques. Cell integrity is compromised and the contents are liberated into a physiological solution and readout using the apparatus and methods described herein.

Through the addition of covalently reactive fluorophores to a biological sample, the protein amongst others are multiply labelled. This can then be incubated in the chamber for static hybridisation to the affinity patches on a sensing surface within the chamber; such as antibodies which can be immobilised to the surface covalently in distinct patches, to different proteins from the sample. The fluorescently labelled or inherently detectable analyte binds and is counted at the single molecule level at each individual affinity patch. Although in one approach a fluorescent dye or other label is provided to label the proteins this can be achieved using label free approaches where fluorescent or other detector properties can be relied on.

In an embodiment, the current single molecule detection platform 108, 110 is centred around a microscope, the Nikon Ti-E, fitted with a perfect focus system (PFS) which is to maintain correct and reproducible focus at the single molecule level, coupled with the need to scan different regions of the slide. Scanning of the slide is accomplished through control of the Z axis of the microscope and control of the XY stage holding the microfluidic device. The locations of the affinity patches 106 are predetermined and so the whole slide does not have to be scanned. This facilitates the analysis of each affinity patch in single molecule accumulation data collection.

By using a highly sensitive camera such as an EMCCD camera with high quantum efficiency from Andor, fluorescently labelled single molecules can easily be resolved. Through the use of the optical sectioning properties of Total Internal Reflection Microscopy (TIRFM), the binding of molecules to an affinity patch on a sensing surface of the hybridisation chamber can be monitored in real time.

Even in low concentrations of analyte, the arrangement according to the invention allows quantification, for example, fixed within a 9.6 nanoliter volume, 16 100 micrometer affinity patches of nanomolar affinity, 90% of the analyte is bound. As discussed in more detail below, with calibrated antibody affinity in the affinity patch and knowledge of the equilibrium point at which the count does not change, the absolute number of molecules for other analyte elements can be obtained from the number of detection events. It will be noted that the affinity patch can take any appropriate form including antibodies, nucleotides, lectins or aptamers as appropriate.

Although in the embodiment shown static hybridisation is introduced, that is, the analyte is in a closed and confined volume, it would be appreciated that in a continuous flow regime analyte can flow past and bind to the affinity regime.

Visualising single molecules is possible owing to the dilution of sample and calculation of maximal density of molecules at the surface. In addition the observation of single and multistep photo bleaching strongly favours the observation of single molecules, in addition to fluorescence intermittency (blinking) of green fluorescent protein (GFP); all of which support the visualisation of single molecules.

An approach to single molecule detection can for example be understood with reference to FIGS. 3 and 4 using total internal reflection (TIRF) microscopy apparatus, available from Nikon. (http://www.nikon.com/products/instruments/lineup/biological-microscopes/application/tirf2/index.htm) With analyte in an aqueous medium, TIRF at the interface with the affinity patch allows specifically immobilised single molecule detection. Only fluorescent molecules which dwell at the surface on the order of the camera exposure time will be counted.

Through the use of TIRF Microscopy and an Electron Multiplying Charged Couple Device (EMCCD) as detector, it is possible to visualise single fluorescent molecules 0-200 nm from the surface of the glass, following excitation of the fluorophore through an evanescent wave produced as a laser beam which totally internally reflects at the glass to aqueous media interface. In conjunction with image analysis this enables exquisite distinction between molecules bound to the surface specifically and molecules transiently binding to the surface. Fluorescent molecules which enter the TIRF illumination volume through Brownian motion do not significantly increase the background fluorescence (and hence lower the signal to noise ratio), due to the high diffusion coefficient of the molecular species, which is on the order of 10̂8 cm2s-1. The limited effective penetration depth of TIRF minimises background fluorescence and illuminates just molecules bound to the surface. Different TIRF angles with different frequencies of light allow different penetration depths.

Refractive index differences between the glass and media (analyte solution) phases govern how light is refracted or reflected at the interface as a function of angle. Beyond a critical angle the light is totally reflected from the interface. The reflection generates an electromagnetic field in the media, the evanescent field, of identical frequency to the reflected light, which exponentically decays as a function of distance from the interface. The exponential decay of the evanescent fiend intensity allows only fluorophotes with extremely high proximity (typically <200 nm) to the coverslip to be excited. The characteristic distance/depth is dependent on wavelength, polarisation and incidence angle of light and refractive index differences at the interface.

In conjunction with a known volume of a chamber in the microfluidic device, proportional to the affinity constant of the analyte and the capture reagent, the direct quantification of the total specific analyte in a sample is enabled. This is owing to the majority of analyte being bound to the surface at equilibrium, made possible through the high affinity of the capture agent to analyte essentially governing the percentage bound to the surface.

Referring particularly to FIG. 3 the bound analyte 300 which typically has a low refractive index is penetrated by an evanescent wave at the specimen interface with the high refractive index cover glass 302. Penetration is typically less than 200 nm. Incident light 304 is reflected at 306 for detection at the protector 110. In an objective based TIRF microscopy, as opposed to prism or waveguide based TIRF (which are equally applicably) an objective lens or lenses 400 as shown in FIG. 4 incident on analyte 402 bound at specimen cover glass 404 is shown. The laser illumination and reflection is shown at 406. Currently a Nikon 1.49NA 60× oil immersion objective is used but many other permutations are possible for single molecule detection, such as a water immersion objective (oil immersion causes trouble with translation of the device over the stage). This format allows rapid and unbiased read out of multiple affinity patches at defined regions of a micro fluidic chip to be read with high signal to noise ratio, to the single molecule level.

The manner in which absolute quantification—in terms of the total amount of individual molecules present in solution—is achieved will now be described. In order to capture the predominant proportion of antigen from solution, the analyte must be incubated in a chamber of minimal volume. Through manipulation of the mass action equation shown below we can approximate the percentage of analyte which will bind to a fixed concentration of antibody in a static chamber originating from a single antibody spot of 100 μm; a spot of such size, with antibodies at approximately 16 nm, perfectly orientated will approximately fit 5×10-17 moles of antibody, which are bivalent and so this gives 1.02E-16M of active binding site per spot. Making an assumption of concentration of antibody [B] in the confined chamber, the dissociation constant of the average antibody Kd at 10-9, we can estimate the ratio of bound (AB) to unbound (A). This makes the assumption that the antibody concentration makes little change at equilibrium from the initial approximation of concentration, owing to the comparatively low analyte levels (such as those found in a single cell).

$$\frac{[ B ]}{[ Kd ]} = \frac{[ AB ]}{[ A ]}$$

The chamber volume is proportional to antibody affinity, allowing the design to allow the technique to be quantitative over the protein content of the sample. The chamber may have an extremely low profile, from 10-50 μm in height and 2-3 mm in diameter. At 3 mm in diameter and 10 μm in height, the volume of the chamber is approximately 1 nl. A single side of the chamber can be coated with 15 100 μm spots with 100 μm spacing between each spot. If the wells/chambers are fabricated onto 150 μm glass coverslips, it is also possible to have antibodies on both sides of the chip.

Through manipulation of the mass action equation and the dimensions of a low aspect ratio microfluidic chamber, with a single sensing surface, containing antibody spots of 100 μm in diameter and 100 μm spacing. Particularly, for use as a biosensor, the height of the chamber can also be reduced so that a greater proportion of the analyte is bound at any one time. An estimation of 5×10-17 moles of bivalent antibody may be present in a single spot to a particular analyte within a confined chamber. Rather than having inter-spot spacing in a square chamber with rows and columns of antibodies to different proteins, it is plausible to print antibodies in a linear arrangement, to reduce wasted space within the chamber.

In the bleaching mode of quantification, by counting the irreversible multiple stepwise bleaching we can be certain that once bleached, the molecule will not be detected again. Hence by allowing the saturated affinity patch at equilibrium with analyte, to dissociate over a time such as the t1/2 for the capture agent and analyte, the bleached molecules will dissociate/exchange and labelled, unread analyte will bind.

$${t}_{1 / 2} = \frac{0.693}{k - 1}$$

This will then be counted and bleached and repeated until all of the analyte is bound and counted over time. If an antibody has a Kd in the range of 10-7 to 10-10 then using the relationship stated above half of a given amount of antibody-antigen complex will dissociate. This is useful as when equilibrium is reached it means we simply have to bleach the molecules already bound to the surface and allow half of the molecules to dissociate after a predetermined period of time and recount the spots on the affinity patch, to count molecules which have previously not been counted. This enables more spots in be used in a larger chamber, where less of the analyte is bound, potentially enabling for example a 256 feature array in a chamber of 20 μm in height, 3.1 mm length and width (192 nL), where approximately 20% of the analyte is bound at any one time. In an embodiment, allowing equilibrium to reach, count and then bleach each hour for five hours, in this instance, allows all available molecules in the system counted and irreversibly bleached in this time frame. In the instance where the antibody off time is great, it may also be possible to effectively extrapolate the equilibrium point through following the early kinetic phase of the hyperbolic curve, with subsequent calculation of the percentage bound and that free in solution. Variability in dissociation rate constants can be taken into account.

FIGS. 7a to 7c show some data obtained in an experiment using the iterative bleaching procedures described herein. The calibration curve shown in FIG. 7a was prepared for single molecule counting using nanoliter chips with defined quantities of analyte read out through single molecule bleaching over time.

The data was obtained by introducing analyte into a microfluidic chamber and waiting for half an hour. The laser was then turned on and successive images of the analyte were taken every few seconds. Each loss of single molecules between consecutive images due to bleaching by the laser was determined by subtracting successive frames and by using a standard single molecule algorithm to identify the features associated with single molecule loss. Therefore both differencing and bleaching were combined to decrement a number of molecules observed in each successive frame.

The total number of molecules decremented during the experiment was then added up to obtain the total molecular count over a period of half an hour. The results as shown in FIGS. 7a to 7c span five orders of magnitude. This demonstrates one of the advantages of the presently-described method, whereby very high dynamic ranges can be achieved. The number of molecules counted is not equal to the number of molecules of analyte put into the chamber for the experiment shown in FIGS. 7a to 7c, but a calibration curve could nonetheless be prepared as shown in the figures.

The manner in which recount is prevented using an alternative, time multiplexing approach for absolute quantification can be understood from FIGS. 5a to 5c. Each of images A, B, C show frames derived from raw video data, demonstrating single molecule accumulation to an antibody affinity patch, which is limited to the field of view. The frames are taken from the beginning, middle and end of the footage, lasting approximately 12 min. In the first frame A, the autofluorescent halo of the antibody patch can clearly be seen.

To enhance the information available from still single molecule images, time lapse image of the accumulation of antigen (fluorophore): antibody complex, were recorded. Through manipulation of the mass action law, the observed kinetics (kobs) can give information on the rate of association and dissociation of the bi-molecular reaction, as well as define the point of equilibrium, simply by following the reaction in the early kinetic phase. Analysis can be performed on the single molecule scale, through the counting of single molecules. There is a constricted volume, so that the concentration of the antibody is approximately that of its Kd and so the majority of the sample binds to the affinity patch rather than in solution.

It will further be seen that in controlling or analysing binding kinetics, the temperature of the chip can be controlled to modulate the binding kinetics.

Absolute quantification of analyte can thus be achieved from a chamber of known proportions, through continual bleaching of fluorophore labelled analyte, until total analyte in confined space has been counted and irreversibly bleached. Alternatively, as discussed above and below, by monitoring accumulation of fluorophore labelled analyte, it is possible to extrapolate kinetics of binding of analyte in fixed static volume, yielding absolute quantity of analyte in the sample. It is also possible to extrapolate accurately the rate of binding with for example low copy number proteins. Time sharing can be implemented between affinity patches to formulate binding curves for the accumulation of analyte to the sensing surface, to determine quantity of a multitude of analytes. It will be noted that analyte and affinity reagents can encompass a broad spectrum of biological agents a broad spectrum of biological agents, such as protein—protein interaction, antibody—antigen, hybridisation oligo and RNA and lectin—glycoprotein.

It will further be noted that affinity patches have been proposed although alternatively the entire surface could be coated with affinity reagent. A multiplexed approach allows the user to capture and quantify multiple analytes from a sample, through mass transfer of analyte to specific affinity patches contained within the static hybridisation chamber. This means there is less affinity reagent specific to the analyte at the sensing surface, and the end result is that less of the analyte is bound to the surface at any one point. The more affinity patches, the larger the chamber and the less analyte is bound, taking it more into the classical ambient analyte regime common to most microarrays. Currently to deposit affinity patches to predefined locations to the sensing surface of the microfluidic device, a microarrayer from Genomic solutions; the Omnigrid microarrayer is used. The use of affinity patches provides more rapid equilibrium. In particular, where restricted volume and small patches are adopted, this allows the rapid formation of equilibrium. Rapid equilibrium in turn ensures that a significant fraction of the analyte can be quickly baind and quantification performed more rapidly. As described above, one method of image analysis enables the extension of the dynamic range of the device, whereby successive images are subtracted so that only new detection events are counted, preventing recount and hence allowing the rate of addition to be calculated past the point where individual molecules can no longer be distinguished.

Through monitoring the addition of analyte to the microscope field of view confined affinity patch, we can formulate binding curves of the analyte to affinity reagent, this is fit to an equation such as the one shown below, which can also be made to incorporate a diffusion—reaction component. The concentration of the analyte in the static hybridisation chamber can then be approximated with great accuracy. This is also directly comparable to the results of the bleaching method described above.

AB_{T}=AB_{MAX}(1−E^{K}obs^{t)}

Time sharing between affinity patches can be achieved to formulate binding curves for the accumulation of analyte to the sensing surface, to determine quantity, to a multitude of analytes. Through the use of a microcope with a system to maintain focus to the single molecule level, within the TIRFM penetration depth of approximately 200 nm, it is possible to move along the sensing surface of the chip, limited only by the dimensions of the chip. For the extrapolation of binding kinetics as described the camera is capable of moving from affinity patch to affinity patch and take images from each patch in a time sharing fashion. A finite number of images is required to effectively produce a binding curve, approximately 10 points along from the kinetic to steady state rate is required. Reproducibility of the stage revisiting affinity patches to perfect pixel overlap, is not required if after each readout of an affinity patch the molecules are counted and bleached, or subtracted from previous images.

It will be seen that the approaches described herein can be applied in relation to any appropriate analyte. In one example, where circulating tumour cells from a first round chemotherapy cancer patient have been isolated by an FDA approved magnetic bead method and 200 cells have been isolated from the patient, it is of clinical relevance to know the phenotype of the cancer cells circulating in the patient's blood stream. Expression levels of signalling proteins controlling the movement and aggressiveness of the cells must be known, so the correct drug to slow the cells can be chosen and the approach can provide this information through absolute quantification.

The invention further allows working on smaller and smaller populations of cells in drug discovery, biomarker identification, clinical samples; it reduces the contamination from unavoidably mixed and unique cell populations, using actual per-cell numbers of proteins, rather than arbitrary and unrelated response units.

In addition applications are available to single cell proteomics, assay diagnostics, quantification of un-amplified mRNA, enzyme linked enzyme-immunosorbant assay (ELISA), biomarker detection even for low copy number proteins and protein-protein interaction identification and detection.

In addition to quantification, more data can be derived from such technology from digitisation of single molecule data into parameters for kinetic analysis of analyte in a chamber in which a significant proportion of the analyte is bound to the surface. The versatility of the setup promotes a lab on a microscope approach’ using a EMCCD of high resolution and quantum efficiency, a facility or source for microfluidic device and tailorable data acquisition and analysis programs.

The application of this technology hence allows the advancement of proteomics technology to single molecule level and enabling absolute quantification of the total analyte in a sample. Current technology relates the level of analyte expression; whether it is protein or mRNA for example, to that of another analyte whose level of expression is approximately consist from cell to cell. Another commonly used methodology is to differentially label one sample to the normal sample and incubate both on the array, with the disadvantage that different affinities of capture agent to analyte are not represented in the data.

The technology described herein has sufficient sensitivity to perform single cell analysis (both mRNA and protein levels) from cells of unique origin, such as stem cells, where phenotypic morphology is dependent on low copy level transcripts and for biological samples which are limited in number and as such cannot sufficiently be investigated with the current state of the art technology. The invention has the capability to reveal unique and unforeseen events within the cell. The technology has the capability to produce analyte snap shots of cells, giving the ability to determine protein profiles, in addition to the noise and variability of such proteins from within a cell.

Full advantage of the capacity to analyse quantities of an analyte found in single cells can be obtained by incorporating the measurement system into a microfluidic device capable of handling, sorting, trapping, and lysing single cells. FIG. 6a shows a single cell trapped in a geometry which permits the stable trapping of a single cell. The trapped cell is stably contained even with high fluid flow rates through the device. FIG. 6b then shows multiple cells trapped in a geometry which permits the stable trapping of many such cells.

A clinical sample such as a blood sample or a needle biopsy will commonly contain a variety of cell types of which only a fraction will be of interest for analysis. Examples of interesting subpopulations include cancer cells and stem cells. A sorting stage is capable of selecting cells of interest by making a measuring a parameter of each cell and physically separating a subpopulation. One possible parameter measurement can be obtained by staining using a fluorescently labelled antibody. The subpopulation of interest may be separated by pressure based switching of a hydrodynamic flow, use of optical tweezers, or dielectrophoresis or other any other suitable force. Such techniques are well known in the field of microfluidic flow cytomtery and cell sorting. However, before now they have not been integrated with a stage capable of performing protein, rna or other molecular analysis of the sorted population at a single cell level. The present embodiments provide for the possibility of trapping one or more cells of interest in the vicinity of the affinity patch. The trapped cell may be a cell which has been identified and separated in a separation stage. The mechanism for cell trapping may be a commonly known physical barrier contained in the microfluidic device capable of filtering the cell out of the flow.

In order to analyse contents of a cell by the present methods liberatio its contents is required. The cell may be fully lysed or lysed to a degree whereby only a particular compartment or compartments are liberated, for example the constituents of the cytosol may be liberated while the nucleus remains intact. There exist many methods to lyse a cell including acoustic, chemical, mechanical, electrical and optical. Ultrafast lysis techniques such as the use of highly focused laser pulses or pulses of high voltage are suitable for applications requiring high temporal resolution such as investigating post-translational modifications.

A specific embodiment of these additional stages integrated into a microfluidic device is now described. The microfluidic device is fabricated from polydimethylsiloxane using soft lithography and sealed with a glass cover slip to facilitate TIRF microscopy. The device is placed on a Nikon Ti-E inverted microscope to facilitate introduction optical exictation and detection of flourescence.

A sample of cells, a subpopulation of which is bound to flourescently labelled antibodies, is introduced to into the device with flow driven by a KDS200 syringe pump. The cells are hydrodynamically focussed to move through a detection volume in which a laser excites flourescence of labelled antibodies. Flourescence is detected by a photomultiplier tube and is used to trigger the openning of a solenoid switch. When the swith is closed cells flow to a waste channel. Openning of the switch allows cells of interest to flow into the region of the chip where cells are trapped, lysed and analyed.

Those cells which have been selected by the sorting stage flow into chambers where they are mechanically trapped within a microfluidic device by the use of solid features which aim to trap a cell which is under flow in a chamber for subsequent optical lysis and “single-molecule” readout. The feature geometry is such that a single cell may be trapped as shown in FIG. 6a but may be altered to trap multiple cells as shown in FIG. 6b. The fluid delivery is stopped and inlet and outlet reversably blocked to prevent unwanted pressure-driven flow, which may disrupt containment of the cell(s) and the cell lysate. The cell is selectively lysed by optical lysis. This is accomplished through the delivery of 6 ns, λ=532 nm Q-switched Nd:YAG laser pulses via a 40×, 0.90 NA objective to a location ˜10 μm above the cell. The laser is focussed to a small spot where localized plasma formation occurs. This results in the generation of a shock wave, followed by generation of a cavitation bubble whose expansion disrupts cells. Typically, this method is used to totally disrupt many cells within a zone of radius proportional to pulse energy. At low pulse energies the plasma membrane may be punctured. Cytosolic constituents are discharged into the chamber and are analysed by the quantitative, single-molecule, TIRF readout of specified proteins which bind to an affinity patch.

The above-described embodiment allows measurement of protein levels by the methods previously described herein.

Importantly for both mRNA and protein analysis from biological samples, according to the presently described embodiments there is no ensemble averaging of cellular populations, which obscures discrete events within cellular niches. Such multiplexed ensemble free measurements on low copy number transcripts is not possible with the current state of the art technology.

Hence the potential applications for the technology are widespread throughout the molecular biology arena and pharmaceutical industry. One such example is where researchers would like to yield information on low copy number mRNA transcripts and proteins from biological sources, to absolute levels, something which to date has not presently been obtainable. This is pertinent as commonly in biological systems such as cell signalling cascades it is the short lived or transient species in low copy numbers which determine the cell function and fate. The technology is capable of for example producing ‘snap shots’ of protein or mRNA profiles, which can be correlated with exogenous stimuli or cell state.

As a result of the arrangements described herein, multiple advantages are achieved including digitised single molecule measurement, absolute quantification—single molecule counting, absolute protein expression profiles, high sensitivity and used for low copy number proteins and low concentration samples as well as high concentration examples. Data can be visually represented and binding events discriminated, specifically bound and randomly diffusing analyte being distinguished. Unlike known approaches for determining analyte parameters in a detection region, the present embodiments do not require drying of the analyte before said determination step. Instead, the determination and detection of an analyte element bound at a binding zone of such a detection region can be preformed when the analyte is at a boundary with liquid phase material. Hence a more flexible and user friendly approach is provided.

It will be appreciated that the approaches described herein can be opposed or interchanged as appropriate and that any appropriate methodology for counting implemented in software or hardware can be implemented as will apparent to the skilled person.

Although the embodiments are principally described above in relation to single molecule detection, providing automatic calibration and additional robustness when it comes to absolute quantification (for example from resistance to background variations), it will be appreciated that any appropriate analyte element can be accommodated.

It will further be appreciated that any appropriate micro fluidics device, affinity patch and detection counting mechanisms can be implemented.

1. A method of determining an analyte parameter for an analyte in a detection region having at least one analyte binding zone, the method comprising detecting presence of an analyte element bound at the binding zone, incrementing or decrementing an analyte element count and preventing recount of said element.

2. A method as claimed in claim 1 in which the analyte element comprises a single molecule.

3. A method as claimed in claim 1 in which the analyte element comprises a biological molecule, such as protein, glycoprotein, post translational modifications of proteins, DNA and mRNA.

4. A method as claimed in claim 1 in which the detection region comprises a confined region.

5. A method as claimed in claim 4 in which the detection region is provided on a micro fluidic device.

6. A method as claimed in claim 1 in which the analyte binding zone comprises an affinity patch.

7. A method as claimed in claim 6 in which a plurality of affinity patches are provided, and presence of an analyte element is detected at each affinity patch.

8. A method as claimed in claim 1 in which the analyte comprises a cell protein.

9. A method as claimed in claim 8 in which the cell is trapped in the detection region and optically or chemical lysed to release the cell protein.

10. The method as claimed in claim 1 in which the analyte parameter comprises the number of analyte elements detected.

11. A method as claimed in claim 1 in which recount is prevented by bleaching a detected analyte element.

12. A method as claimed in claim 1 in which recount is prevented by image comparison.

13. A method as claimed in claim 12 in which the image comparison step comprises a sequential image subtraction step.

14. A method as claimed in claim 1 in which analyte element fluorescence is detected.

15. A method as claimed in claim 1 in which the analyte is presented to the binding zone in one of a static hybridisation or continuous flow regimes.

16. A method as claimed in claim 1 in which the analyte is bound to a liquid phase material at the binding zone.

17. A method of extrapolating concentration of an analyte comprising a method as claimed in claim 1 and further comprising monitoring the rate of binding over time.

18. An analysis device comprising an analyte chamber having at least one analyte binding zone provided.

19. The device as claimed in claim 18 in which the analyte binding zone comprises an affinity patch.

20. A device as claimed in claim 18 comprising a micro fluidic device.

21. An analysis apparatus comprising an analysis device as claimed in claim 18 and further comprising a detector arranged to detect presence of an analyte element bound at the binding zone.

22. An apparatus as claimed in claim 21 further comprising a processor for receiving a signal from said detector indicative of the presence of an analyte element and further arranged to increment an analyte element count upon receipt of said signal.

23. An apparatus as claimed in claim 22 in which the processor is further arranged to prevent recount of said element in an image comparison step.

24. A computer program comprising instructions for implementing the method of claim 1.

25. A computer readable medium for storing the computer program of claim 24.

26. A method of counting single molecules of un-amplified mRNA, detecting a biomarker from biological serum, performing single cell proteomics or monitoring a protein—protein interaction comprising the method of claim 1.

27. A method, device or apparatus substantially as described herein.
